(19) United States
(12) Patent Application Publication
(10) Pub. No.: US 2018/0110718 A1
(43) Pub. Date: Apr. 26, 2018

(54) LOW-VISCOSITY OIL-IN-WATER EMULSIONS FOR COSMETIC APPLICATIONS

(71) Applicant: BASF SE, Ludwigshafen am Rhein (DE)

(72) Inventors: Matthias Hloucha, Köln (DK); Stefanie Seidler, Düsseldorf (DE); Esther Kuesters, Erkrath (DE); Petra Schulte, Köln (DE); Gabriele Strauss, Düsseldorf (DE)

(21) Appl. No.: 15/568,569

(22) PCT Filed: Apr. 18, 2016
(86) PCT No.: PCT/EP2016/058504

§ 371 Date: Oct. 23, 2017

(30) Foreign Application Priority Data
Apr. 28, 2015 (EP) 15165443.1

(51) Int. Cl. A61K 8/92; A61K 8/06; A61K 8/02; A61K 8/37; A61Q 19/10; A61Q 1/14

(52) U.S. Cl. CPC A61K 8/922; A61K 8/062; A61K 8/0241; A61K 8/37; A61K 2800/21; A61Q 19/10; A61Q 1/14; A61K 2800/412; A61K 8/0208

(57) ABSTRACT

The invention relates to the field of oil-in-water emulsions for cosmetics and to low-viscosity O/W emulsions with very small-sized oil droplets, to a method for the preparation thereof and to the use thereof for impregnating textiles and paper for cosmetics.

### FIELD OF THE INVENTION

The invention relates to the field of oil-in-water emulsions for cosmetics and to low-viscosity O/W emulsions with very small-sized oil droplets, to a method for the preparation thereof and to the use thereof for impregnating textiles and paper for cosmetics.

### STATE OF THE ART

In cosmetics, oil-in-water emulsions (hereinafter O/W emulsions) having an elegant, finely-divided appearance, such as bluish-shimmering emulsions with oil droplet sizes below 500 nm, are popular. Such emulsions can be prepared by the so-called phase inversion process (PIT process) by heating selected O/W emulsions with specific non-ionogenic emulsifiers above the phase inversion temperature and subsequent cooling. According to this PIT process, O/W emulsions with large oil droplets are used as a starting point, which undergo a phase reversal by heating into a W/O emulsion, which is reversible, which is why the original O/W emulsion type is again produced by cooling, the so-called PIT emulsion. The PIT emulsions thus obtained are particularly finely divided compared to the O/W emulsions used and exhibit a more homo-geneous oil droplet size distribution.

A method for preparing low-viscosity PIT emulsions is known from international application WO 89/11907, in which polar triglycerides were able to be used as oils for the first time using a special emulsifier mixture of fatty alcohol ethoxylates or partial esters of fatty acids with HLB values of 11 to 12 and co-emulsifiers such as fatty alcohols or partial esters of fatty acids with polyols.

By this process, stable PIT emulsions are obtained having very different viscosities in the range of 3 to 1400 mPas at 20° C. Phase inversion temperatures up to 95° C. are also necessary. In practice, phase inversion temperatures of at most 90° C. are desired due to the water content in the emulsions.

Novel emulsifiers for preparing PIT emulsions or microemulsions are known from international application WO 01/89678 which, compared with the prior art, ensure improved storage stability without gelling and crystallization at high temperatures. The emulsifiers proposed therein comprise partial glycerides having a monoglyceride fraction below 50% by weight, and also alcohol polyglycol ethers and fatty alcohols. The co-emulsifiers listed in a long list include, inter alia, addition products of 1 to 15 mol of ethylene oxide onto castor oil and/or hardened castor oil, and also addition products of 15 to 60 mol of ethylene oxide onto castor oil and/or hardened castor oil. The emulsifier mixtures described are outstandingly suitable for storage at high temperatures, but show a need for improvement in storage in the cold.

Emulsions for the finishing of textiles and paper comprising addition products of 7 mol of ethylene oxide onto hardened castor oil are described in International Application WO2008/067944. The emulsions mentioned therein are of the W/O emulsion type and are also milky white or milky yellow and thus do not have finely divided, homogeneously distributed oil droplets.

The object of the invention was to provide novel O/W emulsions for cosmetics which
have very small oil droplet sizes, i.e. are very finely divided
have a homogeneous distribution of oil droplets in order to effect a homogeneous distribution of the oil droplets during application, in particular the spraying thereof.
have very low viscosities, with viscosities ideally below 300 mPas, in order to ensure good sprayability
are storage-stable even under fluctuating thermal stresses
have the desired characteristics, such as oil droplet sizes and viscosities, in particular even after prolonged storage in the cold
can be readily diluted with water without causing gelling or disrupting the emulsion
can be diluted with water at room temperature so that the customer does not have to warm the emulsion,
can be prepared by the PIT process at temperatures which are ideally below 90° C., preferably below 80° C.
are suitable for various oils, especially for polar oils.

It has been found, surprisingly, that the complex object is achieved by O/W emulsions with a specific emulsifier mixture of various ethoxylated hydrogenated castor oils in a fully selected quantitative ratio.

### DESCRIPTION OF THE INVENTION

The present invention relates to low viscosity O/W emulsions with oil particle diameters of 0.01 to 0.3 μm for cosmetic applications, consisting of

(a) one or more oil(s)

(b) an emulsifier mixture

(c) water

(d) and optionally cosmetic active ingredients and/or auxiliaries,

wherein the emulsifier mixture consists of

b1) addition products of 1 to 15 mol of ethylene oxide onto hydrogenated castor oil and

b2) addition products of 20 to 80 mol of ethylene oxide onto hydrogenated castor oil

in a mass ratio (=emulsifier quotient) δ=mass b2:mass (b1+b2) in the range from 0.3 to 0.8.

The present invention further relates to a method for preparing the same and also the use thereof for impregnating textiles and paper for cosmetics, especially wet wipes.

In the context of the present invention, the term “oil” is used for compounds which are liquid and practically immiscible with water at room temperature (21° C.).

In the present application, the term “low viscosity” is used for compounds whose viscosities are below 300 mPas measured according to Brookfield/RVT/23° C.+/−3° C./Sp 3/50 rpm. They preferably have viscosities in the range from 1 to 200 mPas determined according to Brookfield/RVT/23° C.+/−3° C./Sp 3/50 rpm.

The terms “oil particle diameter or oil droplet diameter” or simply “particle or droplet diameter” are used synonymously in the present application and describe the diameter of the oil droplets in [μm] in the O/W emulsion, determined as the median value of the volume distribution with the apparatus: Beckman Coulter LS-230, small volume module; measurement info/parameters: Mie scattering, using PIDS data, refractive index of water: 1.332, with refractive index of 1.47 for the oil phase. The oil particle diameters of the O/W emulsions according to the invention are in the range from 0.01 to 0.3 μm, preferably in the range from 0.06 to 0.2 μm.

The polarity index is given in the units [mN/m], and is a measure of the polarity of an oil defined as the interfacial tension of the oil versus water and was determined using a ring tensiometer (Krüss K 10) which measures the interfacial tension by analogy with the ASTM method D971-99a, 2004. In general, oils having polarities above 35 mN/m are referred to as non-polar and between 5 and 30 mN/m as polar.

The mass ratio of the emulsifiers is given as the emulsifier quotient δ in the manner typical for those skilled in this field and is a mathematical quantity determined from the ratio of the masses m (calculated as active substance content) of emulsifiers used according to the general relationship δ=mass of emulsifier 1: (mass of emulsifier 1+mass of emulsifier 2).

Oils

In the context of the present invention, one or more oils can be present as component a) of the O/W emulsion according to the invention, i.e. it can be a single oil or mixtures of different oils. The oils may be polar and/or non-polar.

For example, the compound classes mentioned below are suitable: Guerbet alcohols based on fatty alcohols having 6 to 18, preferably 8 to 10 carbon atoms, e.g. 2-ethylhexanol or 2-octyldodecanol; esters of linear or branched, saturated or unsaturated C₆-C₂₄-fatty acids with linear or branched, saturated or unsaturated C₆-C₂₄-fatty alcohols. Examples include hexyl laurate, ethylhexyl stearate, myristyl isostearate, myristyl oleate, cetearyl isononanoate, cetyl isostearate, cetyl oleate, decyl oleate, stearyl isostearate, stearyl oleate, isopropyl myristate, isostearyl myristate, isostearyl palmitate, isostearyl stearate, isostearyl isostearate, isostearyl oleate, oleyl myristate, oleyl isostearate, oleyl oleate, oleyl erucate, erucyl isostearate, erucyl oleate, cococaprylate/caprate. Further suitable esters are, e.g. esters of C₁₈-C₃₈-alkylhydroxycarboxylic acids with linear or branched, saturated or unsaturated C₆-C₂₂-fatty alcohols, esters of linear and/or branched, saturated or unsaturated fatty acids with polyhydric alcohols (such as propylene glycol, dimer diol or trimer triol) and/or Guerbet alcohols, triglycerides or triglyceride mixtures, liquid mono-/di-/triglyceride mixtures, esters of C₆-C₂₂-fatty alcohols and/or Guerbet alcohols with aromatic carboxylic acids, particularly benzoic acid (e.g. Finsolv® TN), esters of C₂-C₁₂-dicarboxylic acids with linear or branched, saturated or unsaturated alcohols having 1 to 22 carbon atoms or polyols having 2 to 10 carbon atoms and 2 to 6 hydroxyl groups. Also suitable are vegetable oils, triglyceride mixtures, substituted cyclohexanes, linear symmetrical or asymmetrical dialkyl carbonates (e.g. Cetiol® CC), Guerbet carbonates based on fatty alcohols having 6 to 18, preferably 8 to 10 carbon atoms, linear or branched, symmetrical or asymmetrical dialkyl ethers having 6 to 22 carbon atoms per alkyl group, such as di-n-octyl ether (Cetiol® OE), ring-opening products of epoxidized fatty acid esters with polyols, hydrocarbons such as paraffin or mineral oils, oligo- or poly-alpha-olefins. The dialkyl carbonates and dialkyl ethers may be symmetrical or asymmetrical, branched or unbranched, saturated or unsaturated and can be prepared by reactions which are well-known from the prior art. Suitable silicone compounds are, for example, dimethylpolysiloxanes, methylphenylpolysiloxanes, cyclic silicones (cyclomethicone) and also amino-, fatty acid-, alcohol-, polyether-, epoxy-, fluoro-, glycoside- and/or alkyl-modified silicone compounds. Also suitable are simethicones, which are mixtures of dimethicones with an average chain length of from 200 to 300 dimethylsiloxane units and hydrated silicates.

In the context of the present invention, preference is given to those oil(s) a) having a polarity index of 15 to 55 [mN/m].

According to a preferred embodiment, polar oils having a polarity index of 15 to 30 mN/m are suitable. Examples of suitable polar oils are mono-, di- and tri-fatty acid esters of glycerol which preferably have 6 to 24 and especially 8 to 18 carbon atoms and may be saturated and/or unsaturated and are obtained by chemical synthesis from a natural (vegetable or animal) source. An example is a triglyceride ester of a C8/C10 fatty acid mixture which is obtainable under the trade name Myritol ®312 from BASF Personal Care & Nutrition GmbH.

Other suitable polar oils are vegetable oils further known in the cosmetic industry such as peanut oil, castor oil, coconut oil, corn oil, olive oil, palm kernel oil, sunflower oil, soya oil, rapeseed oil, almond oil, grape seed oil, thistle oil, wheat germ oil, evening primrose oil, macadamia nut oil, argan oil, avocado oil and the like.

Also suitable as polar oils are the esters of benzoic acid, preferably the benzoic esters of alcohols having 8 to 22 carbon atoms, and in particular the benzoic esters of straight-chain alcohols having 12 to 15 carbon atoms. Especially suitable are benzoic esters of a C12-C15 alcohol mixture which are obtainable, for example, under the trade name Cetiol® AB (INCI: C12-C15 Alkyl Benzoate) from BASF Personal Care & Nutrition GmbH.

Likewise suitable as polar oils are the so-called ester oils, i.e. esters of linear C6-C22 fatty acids with linear or branched C6-C22 fatty alcohols or esters of branched C3-C13-carboxylic acids with linear or branched C6-C22-fatty alcohols or esters of linear C6-C22-fatty acids with branched C3-C13-alcohols. Examples include decyl oleate, 2-ethylhexyl palmitate, isopropyl stearate, isopropyl myristate and cetyl stearyl octanoate.

As medium polarity oils with a polarity index which is generally above 30 to about 35 mN/m, dialkyl carbonates are advantageous, which are preferably derived from fatty acids having 6 to 12 carbon atoms, such as dicaprylyl carbonate.

The oils which are widely known from the cosmetic industry, preferably hydrocarbon-based oils such as aliphatic and/or aromatic oils preferably having 8 to 32, in particular 15 to 20, carbon atoms, can be used as non-polar oils with a polarity index of about over 35 mN/m. Examples are squalane, squalene, paraffin oils, isohexadecane, isoeicosecane, polyolefins such as polydecenes, hydrogenated polyisobutenes, dialkylcyclohexane and mineral oil. Preference is given to paraffin oils, such as the low-viscosity paraffins (paraffinum perliquidum), which have a viscosity of 25 to 80 mPa·s and/or the viscous paraffins (paraffinum subliquidum) which, as an oily liquid, have a viscosity of 110 to 230 mPa·s. Further preferred are the hydrogenated polyisobutenes, which are obtainable, for example, from BASF SE under the trade name Luvitol® Lite.

In the context of the invention, especially preferred are oils selected from the group consisting of triglycerides of C6-C22 fatty acids, hydrogenated polyisobutenes, benzoic esters of alcohols having 8 to 22 carbon atoms and esters of C6-C22 fatty acids with linear C6-C22 fatty alcohols. Particularly suitable are group consisting hydrogenated polyisobutenes, benzoic esters of alcohols having 8 to 22 carbon atoms, triglycerides of C6-C12 fatty acids and esters of C6-C12 fatty acids with linear unsaturated C6-C22 fatty alcohols.

In the context of the invention, especially emphasized as oils are benzoic esters of alcohols having 8 to 22 carbon atoms, and in particular benzoic esters of a C12-C15 alcohol mixture.

The following table lists the polarities of the typical, most frequently used oils. Further oils with their polarity indices are described in DE 102004003436 A1, which are incorporated herein:

| Oil | Polarity index [mN/m] |
|---|---|
| Isoparaffin (C₁₂-C₁₄) | 53.0 |
| Squalane ® | 46.2 |
| Isohexadecane (ARLAMOL ® ND) | 43.8 |
| Mineral oil (paraffin oil perliquidum) | 43.7 |
| Mineral oil (paraffin oil subliquidum) | 38.3 |
| Hydrogenated polyIsobutenes (Luvitol ® Lite) | 44.7 |
| Isoeicosane | 41.9 |
| Dioctylcyclohexanes | 39.0 |
| Cetyl stearyl octanoate | 28.6 |
| Isopropyl myristate | 24.2 |
| 2-Ethylhexyl palmitate | 23.1 |
| Isopropyl stearate | 21.9 |
| Decyl oleate | 18.7 |
| C12-C15 alkyl benzoates | 17.1 |
| Rapeseed oil | 21.9 |
| Capryl/Capric triglycerides | 21.3 |
| Peanut oil | 20.5 |
| Almond oil | 20.3 |
| Sunflower oil | 19.3 |
| Avocado oil | 18.3 |
| Olive oil | 16.9 |
| Castor oil | 13.7 |
| Wheat germ oil | 8.3 |

Emulsifiers

In the context of the present invention, it is essential to use as emulsifiers exclusively the mixture of the emulsifiers b1) and b2). These are addition products of ethylene oxide onto hydrogenated castor oil. Castor oil is obtained from the seeds of the castor oil plant and consists mainly of triglycerides of ricinoleic acid, also called triricinolein, and other fatty acids and some volatile constituents. As a rule, the following fatty acid content is present in the triglycerides:

77-83% by weight ricinoleic acid

3-5% by weight linoleic acid

4-9% by weight oleic acid

1-2% by weight palmitic acid

1-3% by weight stearic acid

and also minor amounts of other fatty acids.

In hydrogenated castor oil, the double bonds of the unsaturated fatty acids have been hydrogenated. A measure for the completeness of the hydrogenation is the iodine number, which is preferably below 2.0 g/100 g according to ISO 3961. Such hydrogenated castor oil is known to those skilled in the art and is commercially available.

Emulsifiers b1) and b2) are addition products of ethylene oxide onto hydrogenated castor oil, wherein the addition of ethylene oxide, as is known to those skilled in the art, corresponds to a statistical distribution and thus gives an average value.

Emulsifier b1) is addition products of 1 to 15 mol of ethylene oxide onto hydrogenated castor oil, preference being given to addition products of 3 to 12 mol, more preferably 5 to 10 mol and, in particular, 7 mol of ethylene oxide onto hydrogenated castor oil, as emulsifiers b1). The product with the INCI name PEG-7 Hydrogenated Castor Oil, with 7 mol of ethylene oxide attached to hydrogenated castor oil, is available from BASF under the name Cremophor® WO 7 Surfactant.

Emulsifier b2) is addition products of 20 to 80 mol of ethylene oxide onto hydrogenated castor oil, also with a statistical distribution of ethylene oxide. Preferred emulsifiers b2) are addition products of 25 to 60 mol, particularly preferably 35 to 60 mol and especially 38 to 45 mol of ethylene oxide onto hydrogenated castor oil. The product with the INCI name PEG-40 Hydrogenated Castor Oil, with 40 mol of ethylene oxide attached to hydrogenated castor oil, is available from BASF under the name Eumulgin® CO 40 and is exceptionally suitable.

Additionally in the context of the invention, the mass ratio of b2:b1, which is typically specified by those skilled in the art as the emulsifier quotient δ=mass b2: mass (b1+b2), is essential and is in the range from 0.3 to 0.8.

The oils a) and the emulsifier mixture b) are preferably in a weight ratio from 1:3 to 3:1, particularly preferably 1:2 to 2:1 and especially 1:1.5 to 1.5:1.

According to one embodiment of the present invention, O/W emulsions with hydrogenated polyisobutenes as a) and an emusifier mixture b) composed of b1) addition products of 7 mol of ethylene oxide onto hydrogenated castor oil and b2) addition products of 38 to 45 mol of ethylene oxide onto hydrogenated castor oil with an emulsifier quotient δ=mass b2): ((b1)+(b2)) in the range from 0.5 to 0.6 exhibit very good properties.

According to a further embodiment of the present invention, O/W emulsions are very advantageous which comprise benzoic esters of alcohols having 8 to 22 carbon atoms as oil a) and an emusifier mixture b) composed of b1) addition products of 7 mol of ethylene oxide onto hydrogenated castor oil and b2) addition products of 38 to 45 mol of ethylene oxide onto hydrogenated castor oil with an emulsifier quotient 6 in the range from 0.3 to 0.8. These are particularly finely divided O/W emulsions with extremely small oil droplet diameters.

As a further necessary constituent, the O/W emulsions comprise water, wherein the amount of water makes up to 100% by weight and is generally between 40 to 80% by weight.

Cosmetic Active Ingredients and Auxiliaries

Cosmetic active ingredients and auxiliaries are optional and depend on the cosmetic application of the O/W emulsions according to the invention.

Typically, at least one auxiliary, such as a preservative and/or pH regulator, is present.

Suitable preservatives include benzoic acid or salts thereof, phenoxyethanol, ethylhexylglycerin, dicaprylyl glycol, formaldehyde solution, parabens, pentanediol, sorbic acid, dehydroacetic acid and/or the silver complexes known under the name Surfacine®, and the further substance classes listed in Annex 6, parts A and B, of the cosmetics directive. As pH regulators it is possible to use the compounds known to those skilled in the art for cosmetics, for example, citric acid or salts thereof such as trisodium citrate, lactic acid or salts thereof, gluconic acid or salts thereof such as sodium gluconate.

The pH regulators are preferably used in such amounts that the pH of the O/W emulsions is in the range from 3 to 7, preferably from 3.5 to 5.5. Buffer systems are particularly popular in this case.

Furthermore, water-soluble thickeners, such as natural and/or synthetic polymers such as xanthan gum, hydroxyethylcellulose, polyvinylpyrrolidone or high molecular weight polyethylene oxides, are often also present as auxiliaries.

Hydrotropes may also be present to improve the flow behavior. Typical examples are glycerol; alkylene glycols such as ethylene glycol, diethylene glycol, propylene glycol, butylene glycol, hexylene glycol and polyethylene glycols having an average molecular weight of 100 to 1000 Dalton; technical-grade oligoglycerol mixtures having a degree of self-condensation of 1.5 to 10 such as technical-grade diglycerol mixtures having a diglycerol content of 40 to 50% by weight; methylol compounds such as, in particular, trimethylolethane, trimethylolpropane, trimethylolbutane, pentaerythritol and dipentaerythritol; lower alkyl glucosides particularly those having 1 to 8 carbon atoms in the alkyl radical such as methyl glucoside and butyl glucoside; sugar alcohols having 5 to 12 carbon atoms such as sorbitol or mannitol, sugars having 5 to 12 carbon atoms such as glucose or sucrose; amino sugars such as glucamine and/or dialcoholamines such as diethanolamine or 2-amino-1,3-propanediol.

Perfume oils are also advantageously present as cosmetic active ingredient and/or auxiliary. Perfume oils include mixtures of natural and synthetic odorants. Natural fragrances are extracts from flowers (lily, lavender, rose, jasmine, neroli, ylang ylang), stems and leaves (geranium, patchouli, petitgrain), fruits (aniseed, coriander, caraway, juniper), fruit peels (bergamot, lemon, orange), roots (mace, angelica, celery, cardamom, costus, iris, calmus), woods (pinewood, sandalwood, guaiac wood, cedarwood, rosewood), herbs and grasses (taragon, lemongrass, sage, thyme), needles and branches (spruce, fir, pine, dwarf-pine), resins and balsams (galbanum, elemi, benzoin, myrrh, olibanum, opoponax). Also suitable are animal raw materials, such as, for example, civet and castoreum. Typical synthetic fragrance compounds are products of the ester, ether, aldehyde, ketone, alcohol and hydrocarbon type. Fragrance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allyl cyclohexyl propionate, styrallyl propionate and benzyl salicylate. The ethers include, for example, benzyl ethyl ether, the aldehydes include, for example, the linear alkanals having 8 to 18 carbon atoms, citral, citronellal, citronellyloxyacetaldehyde, cyclamenaldehyde, hydroxycitronellal, lilial and bourgeonal, the ketones include, for example, the ionones, a-isomethylionone and methyl cedryl ketone, the alcohols include anethol, citronellol, eugenol, isoeugenol, geraniol, linalool, phenylethyl alcohol and terpineol, and the hydrocarbons include primarily the terpenes and balsams. However, preference is given to using mixtures of different fragrances which together produce a pleasant scent note. Essential oils of relatively low volatility, which are mostly used as aroma components, are also suitable as perfume oils, e.g. sage oil, camomile oil, oil of cloves, melissa oil, mint oil, cinnamon leaf oil, linden blossom oil, juniperberry oil, vetiver oil, olibanum oil, galbanum oil, labdanum oil and lavandin oil. Preference is given to using bergamot oil, dihydromyrcenol, lilial, lyral, citronellol, phenylethyl alcohol, a-hexylcinnamaldehyde, geraniol, benzyl acetone, cyclamenaldehyde, linalool, boisambrene forte, ambroxan, indole, hedione, sandelice, lemon oil, mandarin oil, orange oil, allyl amyl glycolate, cyclovertal, lavandin oil, clary sage oil, β-damascone, geranium oil bourbon, cyclohexyl salicylate, vertofix coeur, iso-E-super, Fixolide NP, evernyl, iraldein gamma, phenylacetic acid, geranyl acetate, benzyl acetate, rose oxide, romillat, irotyl and floramat alone or in mixtures.

Polymers

Suitable cationic polymers are, for example, cationic cellulose derivatives, such as a quaternized hydroxyethylcellulose obtainable under the name Polymer JR 400® from Amerchol, cationic starch, copolymers of diallylammonium salts and acrylamides, quaternized vinylpyrrolidone/vinylimidazole polymers, such as Luviquat® (BASF), condensation products of polyglycols and amines, quaternized collagen polypeptides, for example lauryldimonium hydroxypropyl hydrolyzed collagen (Lamequat® L/Grünau), quaternized wheat polypeptides, polyethyleneimine, cationic silicone polymers such as amodimethicones, copolymers of adipic acid and dimethylaminohydroxypropyldiethylenetriamine (Cartaretine®/Sandoz), copolymers of acrylic acid with dimethyldiallylammonium chloride (Merquat® 550/Chemviron), polyaminopolyamides such as described in FR 2252840 A for example and also cross-linked water-soluble polymers thereof, cationic chitin derivatives such as quaternized chitosan, optionally in microcrystalline distribution, condensation products of dihaloalkylene, for example dibromobutane with bisdialkylamines, such as bisdimethylamino-1,3-propane, cationic guar gum such as Jaguar® CBS, Jaguar® C-17, Jaguar® C-16 from Celanese, quaternized ammonium salt polymers such as Mirapol® A-15, Mirapol® AD-1, Mirapol® AZ-1 from Miranol.

Useful anionic, zwitterionic, amphoteric and non-ionic polymers are, for example, vinyl acetate/crotonic acid copolymers, vinylpyrrolidone/vinyl acrylate copolymers, vinyl acetate/butyl maleate/isobornyl acrylate copolymers, methyl vinyl ether/maleic anhydride copolymers and esters thereof, non-crosslinked polyacrylic acids and polyacrylic acids crosslinked with polyols, acrylamidopropyltrimethylammonium chloride/acrylate copolymers, octylacrylamide/methyl methacrylate/tert-butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinylcaprolactam terpolymers and optionally derivatized cellulose ethers and silicones. Further suitable polymers and thickeners are listed in Cosm.Toil. 108, 95 (1993).

Silicone Compounds

Suitable silicone compounds are, for example, dimethyl-polysiloxanes, methylphenylpolysiloxanes, cyclic silicones, and amino-, fatty acid-, alcohol-, polyether-, epoxy-, fluorine-, glycosides- and/or alkyl-modified silicone compounds, which at room temperature may either be liquid or else in the form of a resin. Also suitable are simethicones, which are mixtures of dimethicones with an average chain length of from 200 to 300 dimethylsiloxane units and hydrogenated silicates. A detailed overview of suitable volatile silicones is, furthermore, given by Todd et al. in Cosm.Toil. 91, 27 (1976).

Biogenic Active Ingredients

Biogenic active ingredients are understood to mean, for example, tocopherol, tocopherol acetate, tocopherol palmitate, ascorbic acid, (deoxy)ribonucleic acid and fragmentation products thereof, retinol, bisabolol, allantoin, phytantriol, panthenol, AHA acids, amino acids, ceramides, pseudo ceramides, essential oils, plant extracts and vitamin complexes.

Insect Repellents

Suitable insect repellents are N,N-diethyl-m-toluamide, 1,2-pentanediol or ethyl butylacetylaminopropionate.

The O/W emulsions according to the invention especially preferably comprise cosmetic active ingredients and/or auxiliaries and consist in particular of

15 to 22% by weight benzoic esters of alcohols having 8 to 22 carbon atoms

18 to 26% by weight of an emulsifier mixture b) of

b1) addition products of 7 mol of ethylene oxide onto hydrogenated castor oil and

b2) addition products of 38 to 45 mol of ethylene oxide onto hydrogenated castor oil with an emulsifier quotient 6 in the range from 0.3 to 0.8, and

47 to 76.5% by weight water and

0.2 to 5% by weight cosmetic active ingredients and auxiliaries.

Advantageously, preservatives and/or pH regulators are present as auxiliaries and/or active substances d) in the O/W emulsions according to the invention.

The O/W emulsions according to the invention with their particularly small oil droplet diameters and fine distributions are preferably prepared by the phase inversion process.

The invention therefore further relates to a method for preparing low viscosity O/W emulsions with oil particle diameters from 0.01 to 0.3 μm according to claim 1 by the phase inversion process, wherein an O/W emulsion with particle diameter above 2 μm consisting of

(a) one or more oil(s)

(b) an emulsifier mixture consisting of

b1) addition products of 1 to 15 mol of ethylene oxide onto hydrogenated castor oil and

b2) addition products of 20 to 60 mol of ethylene oxide onto hydrogenated castor oil in a mass ratio δ=mass b2): ((b1)+(b2)) in the range from 0.3 to 0.8

(c) water

(d) and optionally cosmetic active ingredients and/or auxiliaries, used, heated to temperatures in the range from 40° C. to 90° C. and subsequently cooled to room temperature.

The method is preferably conducted at temperatures of 60 to 90° C. Within this temperature range is the so-called PIT temperature, i.e. the temperature range to which the coarsely-divided O/W emulsion produced in the first method step has to be heated in the second method step such that a phase inversion into a W/O emulsion takes place. This temperature range can be determined by monitoring the electrical conductivity of the emulsion as a function of temperature using a Tetracon 325/S 4-electrode conductivity measuring cell from WTW. The temperature range within which the conductivity of the O/W emulsions produced in the first method step declines to values below 0.1 mS/cm is the PIT temperature.

In the context of the invention, the inverted W/O emulsion produced in the second method step is cooled, preferably at cooling rates of 0.5 to 2.5° C. per minute. In this case, a phase reversal takes place again, and the particularly finely divided, low-viscosity, storage-stable O/W emulsions according to the invention having the many advantageous properties of the type described above are obtained.

Commercial Applicability

The uncomplicated nature of the dilutability of the O/W emulsions according to the invention with water is of practical improtance. For instance, it is possible, without having to pass through gel phases, to prepare arbitrarily concentrated dilutions of the O/W emulsions all of which have a low viscosity and can therefore be applied extremely well to different surfaces.

The present invention therefore further relates to the use of the O/W emulsions of the type described for impregnating textiles and paper for cosmetics, especially wet wipes.

The O/W emulsions according to the invention are preferably used in the form of their aqueous dilutions, the O/W emulsions preferably being present in amounts from 0.2 to 10% by weight, based on aqueous dilution.

The umbrella term “textile and paper” is understood to mean various different varieties and articles which may sometimes differ considerably in their fields of application and their nature. For example, tissue papers and/or tissue cloth and/or tissues (referred to below as tissues) are suitable. These may be single- or multi-layered. Generally, the papers have a weight per square meter of 10 to 65, preferably 15 to 30 g, and a density of 0.6 g/cm and less. Examples of tissue papers are toilet papers, paper tissues, facial cleansing wipes, make up wipes, refreshing wipes, household wipes and the like. In addition to the paper-based tissues, corresponding tissue cloths are also suitable which are made from fibrous material or fleece material.

Preference is given to multi-layered tissues. In particular, preference is given to those tissues which have an impermeable and/or semi-permeable barrier layer between the individual layers. The semi-permeable barrier layer can, for example, be designed as a semi-permeable membrane. With such wipes, two or more compositions (optionally after prior dilution) can be applied to a cloth. This may be very particularly preferred in order to effect cleaning with one side of the wipes by means of the composition applied to the cloth. The other side can then be used for rubbing, for example, for drying or optionally a care active ingredient can be applied to the skin.

Furthermore, the cloths may consist of at least 3 layers of tissue treated with compositions (optionally after prior dilution). Advantageously, then, one layer of cloth is formed as a semipermeable membrane between at least two layers of treated cloth in each case. The semipermeable membrane is therefore permeable in the direction of the outer cloth layers. Thus, for example, on the inside, a composition (optionally after prior dilution) may be applied to the innermost layer, which is either immiscible and/or not stable with respect to the composition applied to the outer side. This makes it possible to offer “two-in-one cloths” for cleansing and care. The different coloring of the cloth layers and also the different construction of the cloths from two or more materials, in particular with respect to the absorbency and permeability of the different cloth layers, is also possible.

Furthermore, textile fibers, for example, are suitable both from natural fibers, such as cellulose, silk, wool, regenerated cellulose (viscose, rayon), cellulose derivatives, and textile fibers from synthetic fibers such as polyester, polypropylene, polyethylene terephthalate, polyamide, polyolefin and polyacrylonitrile fibers or mixtures of such fibers. These fibers may be woven or non-woven.

The term “impregnation” encompasses any type of application of the O/W emulsion according to the invention or one of its aqueous dilutions to at least one side of the textile or paper. For this purpose, all known appropriate methods are suitable by means of which liquids can be applied to more or less solid surfaces. Examples include: soaking, coating, sprinkling or spraying, dipping, finishing, stripping, etc. The impregnation in this case may be carried out at room temperature or by exposure to heat. After applying the O/W emulsions or aqueous dilutions thereof, a short drying step can follow.

The O/W emulsions according to the invention are preferably used in the form of an aqueous dilution, wherein further additional cosmetic active ingredients and/or auxiliaries are present, depending on the desired application. These may be the same cosmetic active ingredients and/or auxiliaries which have already been described under d) in connection with the O/W emulsions according to the invention or else further customary active substances and auxiliaries (e) known to those skilled in the art which are applied in aqueous dilutions to textile and paper and are marketed in particular as wet wipes. Therefore, the auxiliaries and/or active substances described under d) are either present in the O/W emulsions according to the invention or are added to the aqueous dilutions. The further additional cosmetic auxiliaries and active ingredients (e) are, in contrast to d), only present in the dilutions.

Examples of the additional auxiliaries and active ingredients e) here are surfactants, self-tanning agents, UV filters, solubilizers, humectants or skin moisturizing agents or also other oils, for example the oils mentioned under a).

For instance, surfactants are typically additionally present as active ingredient and/or auxiliary in formulations for wet wipes for cleansing the skin, oils for wet wipes for care and/or removal of dirt and UV filters or self-tanning agents for wet wipes for protection against UV radiation or for self-tanning.

Surfactants

In the context of the invention, surfactants are amphiphilic substances which effect the removal and dissolution of dirt, a light rinsing off of the dirt and, as desired, foam regulation.

Surfactants present may be anionic, non-ionic, cationic and/or amphoteric or zwitterionic surfactants. In surfactant-containing cosmetic preparations, at least one anionic surfactant is preferably present.

Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partially oxidized alk(en)yl oligoglycosides and glucuronic acid derivatives, fatty acid N-alkylglucamides, protein hydrolyzates (especially wheat-based vegetable products), polyol fatty acid esters, sugar esters, sorbitan esters, polysorbates and amine oxides. If the nonionic surfactants contain polyglycol ether chains, these may have a conventional homolog distribution, but preferably have a narrow homolog distribution.

Zwitterionic surfactants refer to those surface-active compounds which bear at least one quaternary ammonium group and at least one —COO(—)—or —SO3(-)- group in the molecule. Particularly suitable zwitterionic surfactants are the so-called betaines, such as the N-alkyl-N,N-dimethylammonium glycinates, for example cocoalkyldimethylammonium glycinate, N-acylaminopropyl-N,N-dimethylammonium glycinates, for example cocoacylaminopropyldime-thylammonium glycinate, and 2-alkyl-3-carboxylmethyl-3-hydroxyethylimidazoline having in each case 8 to 18 carbon atoms in the alkyl or acyl group, and also cocoacylaminoethyl hydroxyethylcarboxymethylglycinate. A preferred zwitterionic surfactant is the fatty acid amide derivative known under the INCI name Cocamidopropyl Betaine.

Likewise suitable, especially as cosurfactants, are ampholytic surfactants. Ampholytic surfactants are understood to mean those surface-active compounds which, apart from a C8-C18-alkyl or acyl group in the molecule, comprise at least one free amino group and at least one —COOH or —SO3H group and are capable of forming internal salts. Examples of suitable ampholytic surfactants are N-alkylglycines, N-alkylpropionic acids, N-alkylaminobutyric acids, N-alkylimino-dipropionic acids, N-hydroxyethyl-N-alkylamidopropylglycines, N-alkyltaurines, N-alkylsarcosines, 2-alkylaminopropionic acids and alkylaminoacetic acids each having about 8 to 18 carbon atoms in the alkyl group. Particularly preferred ampholytic surfactants are N-cocoalkylaminopropionate, cocoacylaminoethylaminopropionate and C12-18-acylsarcosine.

Typical examples of amphoteric or zwitterionic surfactants are alkyl betaines, alkylamido betaines, aminopropionates, aminoglycinates, imidazolinium betaines and sulfo betaines. The specified surfactants are exclusively known compounds. With regard to the structure and preparation of these substances, reference may be made to relevant review works in this field. Typical examples of particularly suitable mild, i.e. particularly skin-friendly, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefinsulfonates, ethercarboxylic acids, alkyl oligoglucosides and/or mixtures thereof with alkyl oligoglucoside carboxylates, fatty acid glucamides, alkylamidobetaines, amphoacetals and/or protein fatty acid condensates, the latter preferably based on wheat proteins or salts thereof.

Anionic surfactants are characterized by a water-solubilizing, anionic group, for example a carboxylate, sulfate, sulfonate or phosphate group and a lipophilic radical. Skin-compatible anionic surfactants are known to the person skilled in the art in a large number from relevant handbooks and are commercially available. These are especially alkyl sulfates in the form of their alkali metal, ammonium or alkanolammonium salts, alkyl ether sulfates, alkyl ether carboxylates, acyl isethionates, acyl sarcosinates, acyl taurines with linear alkyl or acyl groups having 12 to 18 carbon atoms, and also sulfosuccinates and acyl glutamates in the form of their alkali metal or ammonium salts.

Typical examples of anionic surfactants are soaps, alkylbenzenesulfonates, alkanesulfonates, olefin-sulfonates, alkyl ether sulfonates, glycerol ether sulfonates, a-methyl ester sulfonates, sulfo fatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ethercarboxylic acids and salts thereof, fatty acid isethionates, fatty acid sarcosinates, fatty acid taurides, N-acylamino acids, for example acyl lactylates, acyl tartrates, acyl glutamates and acyl aspartates, alkyl oligoglucoside sulfates, protein fatty acid condensates (especially vegetable products based on wheat) and alkyl (ether) phosphates. If the anionic surfactants comprise polyglycol ether chains, these may have a conventional homolog distribution, but preferably have a narrow homolog distribution.

Usable cationic surfactants are especially quaternary ammonium compounds. Preference is given to ammonium halides, especially chlorides and bromides, such as alkyltrimethylammonium chlorides, dialkyldimethylammonium chlorides and trialkylmethyl-ammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride. In addition, the very readily biodegradable quaternary ester compounds, for example the dialkylammonium methosulfates and methylhydroxyalkyldialkoyloxyalkylammonium methosulfates sold under the trade name Stepantex® and the corresponding products of the Dehyquart° series can be used as cationic surfactants. The term “ester quats” is generally understood to mean quaternized fatty acid triethanolamine ester salts. They can impart an exceptional soft feel to the inventive preparations. These are known substances which are prepared by the relevant methods of organic chemistry. Further cationic surfactants usable in accordance with the invention are the quaternized protein hydrolyzates.

The textiles and papers in the cosmetics sector thus impregnated are preferably used in the field of wet wipes for skin care or skin cleansing (particularly baby care or cleansing). Examples which may be mentioned are care or cleansing wipes for facial skin (so-called facial tissues, make-up tissues/make-up removal etc.), refreshing wipes for skin, antibacterial and/or deodorizing tissues, products for intimate care; such as, for example, tampons, sanitary napkins, panty liners, intimate wipes), moist toilet paper, incontinence products, self-tanning cloths or so-called insect repellent cloths.

For textiles and papers in the cosmetics sector which serve for sun protection or self-tanning, one or more UV light-protection filters or self-tanning agents are preferably present in the O/W emulsions according to the invention diluted with water.

Suitable UV light protection filters are organic substances that are liquid at room temperature or crystalline (light protection filters) which are capable of absorbing ultraviolet rays and releasing the energy absorbed again in the form of longer-wave radiation, for example heat. UV filters may be oil-soluble or water-soluble. Examples of typical oil-soluble UV B filters or broadspectrum UV NB filters include:

3-benzylidenecamphor or 3-benzylidenenorcamphor (Mexoryl SDS 20) and derivatives thereof, e.g. 3-(4-methylbenzylidene)camphor, as described in EP 0693471 B1

3-(4′-trimethylammonium)benzylidenebornan-2-one methylsulfate (Mexoryl SO) 3,3′-(1,4-phenylenedimethine)bis(7,7-dimethyl-2-oxobicyclo[2.2.1]heptane-1-methanesulfonic acid) and salts (Mexoryl SX)

3-(4′-sulfo)benzylidenebornan-2-one and salts (Mexoryl SL) polymer of N-[2(and 4)-(2-oxoborn-3-ylidenemethyl)benzyl]acrylamide (Mexoryl SW)

2-(2H-benzotriazol-2-yl)-4-methyl-6-(2-methyl-3-(1,3,3,3-tetramethyl-1-(trimethylsilyloxy)disiloxanyl)propyl)phenol (Mexoryl XL)

4-aminobenzoic acid derivatives, preferably 2-ethylhexyl 4-(dimethylamino)benzoate, 2-octyl 4-(dimethylamino)benzoate and amyl 4-(dimethylamino)benzoate;

esters of cinnamic acid, preferably 2-ethylhexyl 4-methoxycinnamate, propyl 4-methoxycinnamate, isoamyl 4-methoxycinnamate, 2-ethylhexyl 2-cyano-3,3-phenylcinnamate (octocrylene);

esters of salicylic acid, preferably 2-ethylhexyl salicylate, 4-isopropylbenzyl salicylate, homomenthyl salicylate;

derivatives of benzophenone, preferably 2-hydroxy-4-methoxybenzophenone, 2-hydroxy-4-methoxy-4′-methylbenzophenone, 2,2′-dihydroxy-4-methoxybenzophenone;

esters of benzalmalonic acid, preferably di-2-ethylhexyl 4-methoxybenzmalonate;

triazine derivatives, for example 2,4,6-trianilino(p-carbo-2′-ethyl-1′-hexyloxy)-1,3,5-triazine and 2,4,6-tris[p-(2-ethylhexyloxycarbonyl)anilino]-1,3,5-triazine (Uvinul T 150), as described in EP 0818450 A1, or bis(2-ethylhexyl) 4,4′-[(6-[4-(1,1-dimethylethyl)aminocarbonyl)phenylamino]-1,3,5-triazine-2,4-diyl)diimino]benzoate (Uvasorb® HEB);

2,2-(methylenebis(6-(2H-benzotriazol-2-yl)-4-(1,1,3,3-tetramethylbutyl)phenol) (Tinosorb M);

2,4-bis[4-(2-ethylhexyloxy)-2-hydroxyphenyl]-6-(4-methoxyphenyl)-1,3,5-triazine (Tinosorb S);

propane-1,3-diones, for example 1-(4-tert-butylphenyl)-3-(4′-methoxyphenyl)propane-1,3-dione;

ketotricyclo(5.2.1.0)decane derivatives, as described in EP 0694521 B1;

dimethicodiethyl benzalmalonates (Parsol SLX).

Useful water-soluble UV filters include:

2-phenylbenzimidazole-5-sulfonic acid and the alkali metal, alkaline earth metal, ammonium, alkylammonium, alkanolammonium and glucammonium salts thereof;

2,2-(1,4-phenylene)bis(1H-benzimidazole-4,6-disulfonic acid, monosodium salt) (Neo Heliopan AP)

sulfonic acid derivatives of benzophenones, preferably 2-hydroxy-4-methoxybenzophenone-5-sulfonic acid and salts thereof;

sulfonic acid derivatives of 3-benzylidenecamphor, for example 4-(2-oxo-3-bornylidenemethyl)benzenesulfonic acid and 2-methyl-5-(2-oxo-3-bornylidene)sulfonic acid and salts thereof.

Useful typical UV A filters are especially derivatives of benzoylmethane, for example 1-(4′-tert-butylphenyl)-3-(4′-methoxyphenyl)propane-1,3-dione, 4-tert-butyl-4′-methoxydibenzoylmethane (Parsol® 1789), 1-phenyl-3-(4′-isopropylphenyl)propane-1,3-dione, and enamine compounds, as described in DE 19712033 A1 (BASF), and also hexyl 2-[4-(diethylamino)-2-hydroxybenzoyl]benzoate (Uvinul® A plus).

The UV A and UV B filters can of course also be used in mixtures. Particularly favorable combinations consist of the derivatives of benzoylmethane, e.g. 4-tert-butyl-4′-methoxydibenzoylmethane (Parsol® 1789) and 2-ethylhexyl 2-cyano-3,3-phenylcinnamate (octocrylene) in combination with esters of cinnamic acid, preferably 2-ethylhexyl 4-methoxycinnamate and/or propyl 4-methoxycinnamate and/or isoamyl 4-methoxycinnamate. Combinations of this type are advantageously combined with water-soluble filters, for example 2-phenylbenzimidazole-5-sulfonic acid and the alkali metal, alkaline earth metal, ammonium, alkylammonium, alkanolammonium and glucammonium salts thereof.

Suitable UV light protection filters are especially the substances approved according to Annex VII of the Commission Directive (in the version: Commission Directive 2005/9/EC of Jan. 28, 2005 amending Council Directive 76/768/EEC, concerning cosmetic products, for the purposes of adapting Annexes VII thereof to technical progress), to which reference is explicitly made here.

In addition to the soluble substances mentioned, insoluble light protection pigments, specifically finely dispersed metal oxides and salts, are also useful for this purpose. Examples of suitable metal oxides are especially zinc oxide and titanium dioxide, and additionally oxides of iron, of zirconium, of silicon, of manganese, of aluminum and of cerium, and mixtures thereof. The salts used may be silicates (talc), barium sulfate or zinc stearate. The oxides and salts are used in the form of the pigments for skincare and skin-protecting emulsions, and also for decorative cosmetics. The particles should have a mean diameter of less than 100 nm, preferably between 5 and 50 nm and especially between 15 and 30 nm. They may have a spherical shape, but it is also possible to use those particles which have an ellipsoidal shape or a shape which deviates in some other way from the spherical configuration. The pigments may also be present in surface-treated form, i.e. hydrophilized or hydrophobized. Typical examples are coated titanium dioxides, for example T 805 titanium dioxide (Degussa) or Eusolex® T, Eusolex® T-2000, Eusolex® T Aqua, Eusolex® AVO, Eusolex® T-ECO, Eusolex® T-OLEO and Eusolex® T-S (Merck). Typical examples of zinc oxides are, for example Zinc Oxide neutral, Zinc Oxide NDM (Symrise) or Z-Cote® (BASF) or SUNZnO-AS and SUNZnO-NAS (Sunjun Chemical Co. Ltd.). Suitable hydrophobic coatings are in particular silicones and specifically trialkoxyoctylsilanes or simethicone. In sunscreen compositions, preference is given to using micropigments or nanopigments. Preference is given to using micronized zinc oxide. Further suitable UV light protection filters are given in the overview by P. Finkel in SÖFW-Joumal 122, 8/1996, pp. 543-548 and Parf. Kosm. 80, No. 3/1999, pp. 10 to 16.

In addition to the two aforementioned groups of primary light protection substances, it is also possible to use secondary light protection agents of the antioxidant type, which interrupt the photochemical reaction chain which is triggered when UV radiation penetrates into the skin. Typical examples thereof are amino acids (e.g. glycine, histidine, tyrosine, tryptophan) and derivatives thereof, imidazoles (e.g. urocanic acid) and derivatives thereof, peptides such as D,L-carnosine, D-carnosine, L-carnosine and derivatives thereof (e.g. anserine), carotenoids, carotenes (e.g. alpha-carotene, beta-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. μmol to mol/kg), also (metal) chelating agents (e.g. α-hydroxy fatty acids, palmitic acid, phytic acid, lactoferrin), a-hydroxy acids (e.g. citric acid, lactic acid, malic acid), humic acid, gallic acid, bile extracts, bilirubin, biliverdin, EDTA, EGTA and derivatives thereof, unsaturated fatty acids and derivatives thereof (e.g. gamma-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, superoxide dismutase, zinc and derivatives thereof (e.g. ZnO, ZnSO4), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids), suitable in accordance with the invention, of these specified active ingredients.

Solubilizers

Typical examples of solubilizers are INCI: Polysorbate 20 (Eumulgin® SML 20), INCI: Polysorbate 80 (Eumulgin® SMO 20), INCI: Polysorbate 60 (Eumulgin® SMS 20).

Furthermore, humectants or skin moisturizing agents may be present as auxiliary and/or active ingredient for improving the sensory properties and also moisture regulation of the skin. In addition, it may contribute to improving the penetration capacity of the composition on the wipes.

Suitable according to the invention in particular are, among others, amino acids, pyrrolidonecarboxylic acid, lactic acid and salts thereof, lactitol, urea and urea derivatives, uric acid, glucosamine, creatinine, cleavage products of collagen, chitosan or chitosan salts/derivatives, and especially polyols and polyol derivatives (e.g. glycerol, diglycerol, triglycerol, ethylene glycol, propylene glycol, butylene glycol, erythritol, 1,2,6-hexanetriol, polyethylene glycols such as PEG-4, PEG-6, PEG-7, PEG-8, PEG-9, PEG-10, PEG-12, PEG-14, PEG-16, PEG-18, PEG-20), sugar and sugar derivatives (inter alia fructose, glucose, maltose, maltitol, mannitol, inositol, sorbitol, sorbityl silanediol, sucrose, trehalose, xylose, xylitol, glucuronic acid and salts thereof), ethoxylated sorbitol (sorbeth-6, sorbeth-20, sorbeth-30, sorbeth-40), honey and hardened honey, hardened starch hydrolyzates and also mixtures of hardened wheat protein and PEG-20 acetate copolymer. In accordance with the invention, preference is given to glycerol, diglycerol and triglycerol as suitable humectants.

The O/W emulsions according to the invention diluted with water preferably comprise the additional further cosmetic auxiliaries and/or active ingredients d) and/or e) in an amount from 0 to 40% by weight, preferably from 0.05 to 30% by weight, in particular from 0.05 to 20% by weight, preferably from 0.1 to 15% by weight and especially from 0.1 to 10% by weight, based on the total weight of the O/W emulsion diluted with water.

The O/W emulsions according to the invention are particularly preferably used in the form of their aqueous dilutions to impregnate wet wipes, i.e. give moist textile or paper products.

The present invention therefore further relates to wet wipes for cosmetics, impregnated with an aqueous dilution of an O/W emulsion according to the invention.

### EXAMPLES

The following commercial products of BASF SE and BASF Personal Care & Nutrition GmbH were used:

Eumulgin® CO 60=hydrogenated castor oil ethoxylated with 60 mol of ethylene oxide; INCI: PEG-60 Hydrogenated Castor Oil

Eumulgin® CO 40=hydrogenated castor oil ethoxylated with 40 mol of ethylene oxide; INCI: PEG-40 Hydrogenated Castor Oil

Cremophor® WO 7=hydrogenated castor oil ethoxylated with 7 mol of ethylene oxide; INCI: PEG-7 Hydrogenated Castor Oil

Cetiol® AB=benzoic ester of a C12-15 alcohol mixture; INCI: C12-15 Alkyl Benzoate

Luvitol® Lite=hydrogenated polyisobutenes according to example 3 of WO95/14647 using the catalyst described in example 1 of WO95/14647, fraction 1c obtained after fractionation composed of 7% C12 oligomerization product, 70% C16 oligomerization product and 17% C20 oligomerization product and also higher homologs according to GC analysis; INCI: Hydrogenated Polyisobutene

Myritol® 312=INCI: Caprylic/Capric Triglyceride

Cetiol® V=INCI: Decyl Oleate

### Examples 1 to 6

### Preparation of O/W Emulsions According to the Invention by the PIT Process

To prepare the O/W emulsions according to the invention, the entire amount of water was initially charged and heated to 40° C. With moderate stirring, sodium benzoate, Eumulgin® CO 40 or 60 and citric acid solution were added in succession and stirred until everything was clearly dissolved. Cremophor® WO 7 and the oil components were stirred in successively to the clear solution according to the figures in Table 1. The resulting O/W emulsions were heated with stirring until complete phase inversion and were then cooled to room temperature (23° C.) at a cooling rate of 1 to 2° C. per minute. At 30° C., the final pH was adjusted with citric acid solution to 4.0 or 3.5. On cooling, low-viscosity, storage-stable O/W emulsions having the properties listed in Table 1 were obtained by phase reversal.

The phase inversion temperature is the arithmetic mean of the temperature range starting with the temperature at which the conductivity decreases up to the temperature at which the conductivity is equal to zero. The conductivity is measured using a Tetracon 325/S 4-electrode conductivity measuring cell from WTW.

The viscosity (in mPas) was determined according to Brookfield/RVT/23° C.+/−3° C./Sp 3/50 rpm.

The oil droplet diameter in [μm] was determined as the median value of the volume distribution using the instrument: Beckman Coulter LS-230, small volume module; measurement info/parameters: Mie scattering, using PIDS data, refractive index of water: 1.332; refractive index of the particles: 1.47.

The relevant δ value according to the invention was calculated as=mass of (Eumulgin® CO40 or 60): (mass of (Eumulgin® CO40 or. 60)+mass of (Cremophor® WO7)). The resulting products were stored for 4 weeks at −5° C., 5° C., room temperature (23° C.) and 40° C. The formulations were unchanged in viscosity and appearance.

TABLE 1
Example formulations

| | | Example | Example | Example | Example | Example | Example |
|---|---|---|---|---|---|---|---|
| | | 1 | 2 | 3 | 4 | 5 | 6 |
| | | % by | % by | % by | % by | % by | % by |
| Product | INCI | weight | weight | weight | weight | weight | weight |
| Cetiol ® AB | C12-15 Alkyl | 10.0 | 20.0 | | | | |
| | Benzoate | | | | | | |
| Luvitol ® Lite | Hydrogenated | 10.0 | | 20.0 | 20 | | |
| | Polyisobutene | | | | | | |
| Cetiol ® V | Decyl oleate | | | | | 20 | |
| Myritol ® 312 | Caprylic/Capric | | | | | | 20 |
| | Triglyceride | | | | | | |
| Eumulgin ® | PEG-40 | 11.5 | 11.5 | 11.5 | 11.5 | 9.5 | 6.6 |
| CO 40 | Hydrogenated | | | | | | |
| | Castor Oil | | | | | | |
| Cremophor ® | PEG-7 | 10.5 | 10.5 | 10.5 | 8.5 | 12.5 | 15.4 |
| WO 7 | Hydrogenated | | | | | | |
| | Castor Oil | | | | | | |
| Citric acid | Citric Acid | ca. 1.5 | ca. 1.5 | ca. 1.8 | ca. 1.5 | ca. 1.5 | ca. 1.5 |
| (50% by weight | | | | | | | |
| solution) | | | | | | | |
| Sodium | Sodium | 0.5 | 0.5 | 0.5 | 0.5 | 0.5 | 0.5 |
| benzoate | Benzoate | | | | | | |
| Water demin. | | to 100 | to 100 | to 100 | to 100 | to 100 | to 100 |
| pH | | 4.0 | 4.0 | 3.5 | 3.5 | 4.0 | 4.0 |
| PIT phase | | 67 | 71 | 69 | 76 | 69 | 71 |
| inversion | | | | | | | |
| ternperature | | | | | | | |
| [° C.] | | | | | | | |
| δ = | | 0.52 | 0.52 | 0.52 | 0.58 | 0.43 | 0.30 |
| Appearance | | bluish | bluish | bluish | bluish | bluish | bluish |
| Particle diameter | | 0.094 | 0.076 | 0.089 | 0.089 | 0.087 | 0.080 |
| [μm] | | | | | | | |
| Viscosity | | 130 | 80 | 190 | 80 | 84 | 54 |
| [mPas]: | | | | | | | |
| Stability after | | | | | | | |
| 2 weeks' | | | | | | | |
| storage at | | | | | | | |
| −5° C. | | + | + | + | + | + | + |
| RT | | + | + | + | + | + | + |
| 40° C. | | + | + | + | + | + | + |

### Examples 7 to 10 and comparative examples 1 and 2

The significance of the emulsifier ratio δ as mass of Eumulgin® CO40 or 60: (mass of Eumulgin® CO40 or 60+mass of Cremophor® WO7) with Cetiol® AB as oil is shown in Table 2. It is evident that at δ less than 0.3 there is no phase inversion and a W/O emulsion is present (comparative example 1). At δ=0.9, there is no phase inversion up to 90° C. since the PIT temperature is too high (comparative example 2).

TABLE 2
Formulations

| | | Example | Example | Example | Example | | |
|---|---|---|---|---|---|---|---|
| | | 7 | 8 | 9 | 10 | cf. ex. 1 | cf. ex. 2 |
| | | % by | % by | % by | % by | % by | % by |
| Product | INCI | weight | weight | weight | weight | weight | weight |
| Cetiol ® AB | C12-15 Alkyl | 20.0 | 20.0 | 20 | 20 | 20 | 20 |
| | Benzoate | | | | | | |
| Eumulgin ® | PEG-40 | 9.5 | 14.0 | 16.0 | | 6.0 | 19.8 |
| CO 40 | Hydrogenated | | | | | | |
| | Castor Oil | | | | | | |
| Eumulgin ® | PEG-60 | | | | 9.5 | | |
| CO 60 | Hydrogenated | | | | | | |
| | Castor Oil | | | | | | |
| Cremophor ® | PEG-7 | 12.5 | 8.0 | 6.0 | 12.5 | 16.0 | 2.2 |
| WO 7 | Hydrogenated | | | | | | |
| | Castor Oil | | | | | | |
| Citric acid | Citric Acid | ca. 1.5 | ca. 1.5 | ca. 1.5 | ca. 1.5 | ca. 1.5 | ca. 1.5 |
| (50% by | | | | | | | |
| weight | | | | | | | |
| solution) | | | | | | | |
| Sodium | Sodium | 0.5 | 0.5 | 0.5 | 0.5 | 0.5 | 0.5 |
| benzoate | Benzoate | | | | | | |
| Water demin. | | to 100 | to 100 | to 100 | to 100 | to 100 | to 100 |
| pH | | 4.0 | 4.0 | 4.0 | 4.0 | 4.0 | 4.0 |
| PIT phase | | 66 | 78 | 84 | 82 | No PIT, | No inversion |
| inversion | | | | | | W/O | up to 90° C. |
| temperature | | | | | | emulsion | |
| [° C.] | | | | | | | |
| δ = | | 0.43 | 0.64 | 0.73 | 0.43 | 0.27 | 0.90 |
| Appearance | | bluish | bluish | bluish | bluish | white | white, |
| | | | | | | | creamed |
| Viscosity | | 80 | 110 | 120 | 102 | 330 | Not |
| [mPas]: | | | | | | | determinable; |
| | | | | | | | sample |
| | | | | | | | separates |
| Particle | | 0.087 | 0.076 | 0.074 | 0.078 | >2.0 | >2.0 |
| diameter [μm] | | | | | | | |

### Example 11

### Use of the O/W Emulsions According to the Invention as a Sprayable Wet Wipe Formulation

The O/W emulsion according to the invention according to example 2 was diluted at room temperature without difficulty for the application (figures in % by weight):

| | |
|---|---|
| O/W emulsion of example 2 | 2.0 |
| Water | to 100 |
| pH | 5.5 |

The emulsions remained stable during the dilution; could be diluted with water at room temperature and are immediately usable and sprayable. The dilutions do not gel and are still of low viscosity (<200 mPas).

1. A low viscosity O/W emulsion with oil particle diameters of 0.01 to 0.3 μm for cosmetic applications, consisting of
(a) one or more oil;
(b) an emulsifier mixture;
(c) water;
(d) and optionally cosmetic active ingredients and/or auxiliaries,
wherein the emulsifier mixture consists of b1) addition products of 1 to 15 mol of ethylene oxide onto hydrogenated castor oil and b2) addition products of 20 to 80 mol of ethylene oxide onto hydrogenated castor oil in a mass ratio (=emulsifier quotient) δ=mass b2: mass (b1+b2) in the range from 0.3 to 0.8.

2. The O/W emulsion according to claim 1, wherein b1) are addition products of 3 to 12 mol of ethylene oxide onto hydrogenated castor oil.

3. The O/W emulsion according to claim 1, wherein b2) are addition products of 25 to 60 mol of ethylene oxide onto hydrogenated castor oil.

4. The O/W emulsion according to claim 1, wherein the one or more oil (a) has a polarity index of 15 to 55 [mN/m].

5. The O/W emulsion according to claim 1, wherein the one or more oil (a) are selected from the group consisting of triglycerides of C6-C22 fatty acids, hydrogenated polyisobutenes, benzoic esters of alcohols having 8 to 22 carbon atoms, and esters of C6-C22 fatty acids with linear C6-C22 fatty alcohols.

6. The O/W emulsion according to claim 1, wherein the oil particle diameter is in the range from 0.06 to 0.2 μm.

7. The O/W emulsion according to claim 1, wherein said emulsions have viscosities in the range from 1 to 300 mPas determined according to Brookfield/RVT/23° C.+/−3° C./Sp 3/50 rpm.

8. The O/W emulsion according to claim 1, wherein the one or more oil (a) and the emulsifier mixture (b) are present in a weight ratio of from 1:3 to 3:1.

9. The O/W emulsion according to claim 1, wherein the one or more oil (a) are benzoic esters of alcohols having 8 to 22 carbon atoms and the emulsifier mixture (b) consists of b1) addition products of 7 mol of ethylene oxide onto hydrogenated castor oil and b2) addition products of 38 to 45 mol of ethylene oxide onto hydrogenated castor oil.

10. The O/W emulsion according to claim 1, wherein said emulsions consist of
(a) 15 to 22% by weight benzoic esters of alcohols having 8 to 22 carbon atoms
(b) 18 to 26% by weight of an emulsifier mixture of
b1) addition products of 7 mol of ethylene oxide onto hydrogenated castor oil and
b2) addition products of 38 to 45 mol of ethylene oxide onto hydrogenated castor oil with an emulsifier quotient δ in the range from 0.3 to 0.8,
(c) 47 to 76.5% by weight water,
and 0.2 to 5% by weight cosmetic active ingredients and auxiliaries.

11. The O/W emulsion according to claim 1, wherein the cosmetic active ingredients and/or auxiliaries (d) are preservatives and/or pH regulators.

12. A method for preparing low viscosity O/W emulsions with oil particle diameters from 0.01 to 0.3 μm according to claim 1 by a phase inversion process, wherein an O/W emulsion with particle diameter above 2 μm consisting of
(a) one or more oil;
(b) an emulsifier mixture consisting of
b1) addition products of 1 to 15 mol of ethylene oxide onto hydrogenated castor oil and
b2) addition products of 20 to 80 mol of ethylene oxide onto hydrogenated castor oil in a mass ratio δ=mass b2: mass (b1+b2) in the range from 0.3 to 0.8;
(c) water;
(d) and optionally cosmetic active ingredients and/or auxiliaries,
is heated to temperatures in the range from 40° C. to 90° C. and is subsequently cooled to room temperature.

13. A method for impregnating textiles and paper for cosmetics comprising the use of an O/W emulsion according to claim 1.

14. The method according to claim 13, wherein said emulsion is used in an amount from 0.2 to 10% by weight in the form of an aqueous dilution.

15. A wet wipe for cosmetic use impregnated with an aqueous dilution of an O/W emulsion according to claim 1.

16. The O/W emulsion according to claim 2, wherein b1) are addition products of 5 to 10 mol ethylene oxide onto hydrogenated castor oil.

17. The O/W emulsion according to claim 16, wherein b1) are addition products of 7 mol of ethylene oxide onto hydrogenated castor oil.

18. The O/W emulsion according to claim 3, wherein b2) are addition products of 35 to 60 mol of ethylene oxide onto hydrogenated castor oil.

19. The O/W emulsion according to claim 18, wherein b2) are addition products of 38 to 45 mol of ethylene oxide onto hydrogenated castor oil.
